(19) United States
(12) Patent Application Publication
(10) Pub. No.: US 2025/0204803 A1
(43) Pub. Date: Jun. 26, 2025

(54) Device and method for measuring at least one physiological parameter

(71) Applicant: CENTRE HOSPITALIER UNIVERSITAIRE DE TOULOUSE, TOULOUSE (FR)

(72) Inventor: Eric SCHMIDT, TOULOUSE (FR)

(21) Appl. No.: 18/852,133

(22) PCT Filed: Mar. 27, 2023
(86) PCT No.: PCT/EP2023/057858

§ 371 Date: Sep. 27, 2024

(30) Foreign Application Priority Data
Mar. 28, 2022 (FR) 2202772

(51) Int. Cl. A61B 5/03

(52) U.S. Cl. CPC A61B 5/032; A61B 5/036

(57) ABSTRACT

A measurement device (100) for measuring at least one physiological parameter has a housing (101) which has a measurement chamber (104), the measurement chamber having an internal volume filled with a gas. The measurement device has a sensitive cell (105) permitting measurement of at least a first physiological parameter, the sensitive cell (105) being disposed in contact with the gas contained in the internal volume of the measurement chamber (104). The measurement device has an inlet (107) for a physiological liquid of a patient, the inlet (107) being disposed in such a way that the physiological liquid comes into contact with the gas, and the gas remains interposed between the sensitive cell (105) and the physiological liquid of the patient in such a way as to generate an interface between the gas, contained in the internal volume, and the physiological liquid.

### TECHNICAL FIELD

The invention relates to a device and a method for measuring at least one physiological parameter. The invention applies in particular to measuring the pressure of a physiological liquid drawn off from inside a patient's body. In particular, the invention relates to a device and a method for measuring the pressure of a physiological liquid which is in contact with the central nervous system. More particularly, the invention measures the pressure of the cerebrospinal fluid (CSF) drawn off for example by means of a lumbar puncture.

Intracranial pressure corresponds to the pressure of the CSF present inside the skull in which the central nervous system is bathed. Access to the CSF by lumbar puncture was described by Quincke in 1891. CSF pressure measured by lumbar puncture is also a measurement of intracranial pressure [Lenfeldt N, Koskinen L O, Bergenheim A T, Malm J, Eklund A. CSF pressure assessed by lumbar puncture agrees with intracranial pressure. Neurology. 2007; vol. 68, No 2, pp. 155-158]. The measurement of intracranial pressure by lumbar puncture marks an important advance in neurology in the understanding and especially the identification of intracranial hypertension threatening the central nervous system.

CSF pressure can be measured either statically or dynamically. The static or hydrostatic approach consists in measuring the steady-state pressure and pulsatility of the CSF secondary to cardiac and respiratory cyclic oscillations, whereas the dynamic or hydrodynamic approach consists in applying a known stress to the intracranial fluid system, called a perfusion test, and analyzing the response of the system to this stress [Czosnyka M, Pickard JD. Monitoring and interpretation of intracranial pressure. Journal of Neurology, Neurosurgery & Psychiatry, 2004, vol. 75, No 6, pp. 813-821].

The hydrostatic approach makes it possible to measure the fluid pressure inside the skull and to identify normal pressure and increased pressure known as intracranial hypertension. This can manifest itself through headaches, nausea or vomiting, cognitive disorders and even coma in the most serious cases.

The causes of intracranial hypertension syndromes can be multiple: expansive pathology such as a tumor, intracerebral hematoma or hydrocephalus, but also a blockage of the venous drainage system by thrombophlebitis, etc.

There are also what are referred to as “secondary” causes of intracranial hypertension such as endocrinopathies (Addison's disease, Cushing's disease, hypothyroidism, hyperparathyroidism), a vitamin A deficiency altering the structure of the arachnoid villi, during drug intake (hormonal, antibiotic, lithium or cimetidine), and various other pathologies (sleep apnea syndrome, chronic renal failure, iron deficiency anemia).

The hydrodynamic approach may be attained by injecting physiological serum into the CSF using the access provided by lumbar puncture. This injection of physiological serum applies a stress to the intracranial fluid system. The response of the intracranial system following the perfusion test allows the extraction of various relevant measurements including the measurement of CSF flow inside the skull. Increased flow resistance is an important parameter in clinical practice, as it makes it possible to demonstrate an objective alteration of CSF circulation indicative of hydrocephalus.

### PRIOR ART

In the 1930s, Professor Henri CLAUDE developed a manometer that includes a flexible tube that connects directly to a lumbar puncture needle. The concept of the CLAUDE Manometer is to place the cerebrospinal fluid directly in contact with the measuring area of the manometer. CLAUDE's manometer is now banned for health reasons. Furthermore, CLAUDE's manometer does not make it possible to measure the pulsatility of intracranial pressure.

There is currently no simple and reliable device on the market for quantifying intracranial pressure through measurement of the pressure of the CSF. Thus, practitioners still, in 2022, measure cerebrospinal fluid pressure by measuring a water column as described by QUINCKE in 1891. To this end, practitioners use a seamstress's tape measure laid manually along the single-use sterile flexible tube. In practice, the flexible tube is connected to the lumbar puncture needle. Although this rudimentary method may be sufficient to approximately measure steady-state hydrostatic intracranial pressure, it does not allow accurate measurement of the mean value and the pulsatility of the intracranial pressure, nor does it make it possible to record the signal, or to carry out an exploration of the hydrodynamics.

However, there are more complex solutions which notably use a piezoelectric sensor. The piezoelectric sensor allows liquid pressure to be converted into an electrical signal; the electrical signal must then be amplified and digitized to be processed by specific software. In practice, the sensitive cell of the piezoelectric sensor is placed directly in contact with the CSF in order to measure its pressure using an approach that can be hydrostatic or hydrodynamic.

Generally speaking, the CSF pressure measured by the sensitive cell of the piezoelectric sensor is the summation of two forces per unit area: the pressure transmitted by the fluid, and the pressure linked to the movement of the mass of the fluid. To be specific, this mass undergoes acceleration either directly or due to gravity. The piezoelectric sensor in fact measures the pressure transmitted by the CSF but also the acceleration of the liquid mass which constitutes artifacts and impairs the quality of the CSF pressure signal. Thus a liquid pressure sensor measures all physical acceleration linked to the medical environment in which the pressure measurement is carried out. For example, vibration of medical equipment will generate an artifact in the signal measured by the pressure sensor, as will an impact on the patient's bed, etc. This vibration and other artifacts disrupt delicate measurements such as the pulsatility of intracranial pressure.

The system known as “ICM+”® was developed by the University of Cambridge (United Kingdom) to enable intracranial multi-modal monitoring. This software allows, among other things, measurement of CSF pressure in order to carry out both a static and dynamic analysis of intracranial pressure. The ICM+ system enables reliable measurement of intracranial pressure at the patient's bedside. However, this is software designed for clinical research, and it is not easily accessible to most practitioners at the patient's bedside. In particular, no specific sensor has been designed for the “ICM+”® system.

Document U.S. Pat. No. 4,858,619 describes a system that measures changes in CSF pressure while allowing for the drainage of the CSF. This system allows CSF pressure to be measured remotely, whether the puncture site is lumbar or cranial. In this document, the pressure sensor converts the liquid pressure it measures into an electrical signal. To this end, the sensitive cell of a pressure sensor is placed in a measuring chamber which is first emptied of air and filled with physiological serum by purging. The CSF is then in fluid communication with the measuring chamber filled with physiological serum. On the one hand, the sensitive cell measures physical acceleration artifacts affecting the pressure sensor as well as acceleration of the mass of the physiological liquid, and on the other hand, this system does not allow measurement of CSF pressure pulsatility using a hydrodynamic approach.

Document CN 105641758 describes another CSF drainage system wherein fluid is drawn off directly from the patient's skull. A tube drains the CSF drawn off at a first lower end of a vertically oriented bulb in which a float is arranged. The bulb has a second upper end connected to a guide tube which is equipped with a pressure sensor. The guide tube is filled with a gas. The float thus constitutes a membrane with the aim of separating the CSF located under the float and the gas located above the float and in the guide tube. The purpose of this system is to prevent liquid from coming into contact with the pressure sensor, which may then not need to be sterilized. The system thus measures the pressure of the CSF through the gas contained in the guide tube. When the CSF pressure increases above a certain threshold, a solenoid valve allows the CSF to drain into a collection bag. However, the monitoring of changes in CSF pressure is not reliable. To be specific, the measurement of the changes in CSF pressure is performed by measuring the pressure of the gas contained in the guide tube even though the gas is not in direct contact with the CSF. In addition, the float that separates the gas from the CSF is likely to disrupt the measurements by damping the changes in CSF pressure but also by the acceleration that the float can undergo in the event of movement of the tubing or the bulb

The invention aims to overcome all or some of the aforementioned drawbacks.

### SUMMARY OF THE INVENTION

The invention aims to provide a reliable and cost-effective solution for measuring at least one physiological parameter such as CSF pressure by lumbar puncture.

The invention aims to reliably measure at least one physiological parameter at the patient's bedside.

In particular, the invention aims to provide a technical solution for reliably measuring changes in CSF pressure over time.

The invention aims in particular to reliably measure the steady-state pressure or the pulsatile pressure of the CSF.

To this end, a first aspect of the invention concerns a measuring device for measuring at least one physiological parameter. The measuring device includes a housing that extends longitudinally between a first end and a second end opposite the first end. The housing comprises three parts:
a measuring chamber which is arranged in the housing, the measuring chamber having an internal volume which is filled with gas,
a sensitive cell making it possible to measure at least a first physiological parameter, the sensitive cell being arranged in contact with the gas contained in the internal volume of the measuring chamber, and
an inlet for the intake of a physiological liquid of the patient into the measuring chamber, the inlet being arranged such that the physiological liquid comes into contact with gas and the volume of gas is interposed between the sensitive cell and the physiological liquid of the patient.

According to the invention, the measuring chamber makes it possible to generate two interfaces: a first interface between the gas contained in the internal volume and the physiological liquid of the patient which enters through the inlet, a second interface between the gas contained in the measuring chamber and the sensitive cell.

The measuring device according to the invention makes it possible to carry out reliable measurements of a liquid physiological parameter such as CSF pressure. Indeed, in a fluid, according to Pascal's principle, changes in pressure at one point are transmitted in full to all points of this fluid. The changes in pressure of the physiological liquid are therefore transmitted to the sensitive cell of the sensor via a gas bubble, which corresponds to a “hydropneumatic transmission interface” or a liquid/gas interface.

Furthermore, the device according to the invention makes it possible to reduce artifacts due to vibration of the sensor.

In embodiments of the first aspect of the invention, the measuring chamber may have an internal volume that is non-zero and less than or equal to 1 ml, in particular, the internal volume must be strictly less than 1 ml, preferably less than 0.7 ml. The internal volume of the measuring chamber may thus contain a volume of gas making it possible to control the damping of the pulsatility of the pressure of the physiological liquid. This is so that the damping of pulsatility is acceptable in clinical practice.

In embodiments, the gas contained in the internal volume of the measuring chamber may correspond to atmospheric air. To be specific, since the method for manufacturing the measuring device, for example by plastic injection molding, is not carried out under vacuum, the measuring chamber is naturally filled with atmospheric air.

In embodiments of the first aspect of the invention, the measuring chamber may have an internal volume of between 0.2 ml and 0.5 ml.

In embodiments of the first aspect of the invention, the sensitive cell may be a piezoelectric cell of a pressure sensor. The liquid/gas interface also makes it possible to dampen the physical acceleration experienced by the pressure sensor. This improves the reliability of the measurements.

In embodiments of the first aspect of the invention, the housing may comprise a sealing membrane which is interposed between the sensitive cell and the internal volume of the measuring chamber, the sealing membrane protecting the sensitive cell. The sensitive cell of the sensor may be, for example, a piezoelectric cell of a pressure sensor. The sealing membrane makes it possible to isolate the CSF and, more generally, the patient's biological tissue from the highly toxic electronic components that a piezoelectric sensitive cell may include.

In embodiments of the first aspect of the invention, the internal volume of the measuring chamber may be cylindrical. A cylindrical shape makes it possible to form a liquid/gas interface when the physiological liquid enters the measuring chamber.

In embodiments of the first aspect of the invention, the measuring device may comprise a connector arranged at the inlet of the housing, this connector sealingly connecting the measuring chamber to a connection device for connection to the physiological liquid of the patient. The physiological liquid connection device may be a lumbar puncture needle. Preferably, the lumbar puncture needle is connected directly to the connector of the measuring device.

In embodiments of the first aspect of the invention, the inlet may be openable and closable to control the entry of physiological liquid into the measuring chamber.

In embodiments of the first aspect of the invention, the measuring device may comprise transmission means for transmitting measurements of the first physiological parameter to a remote digital terminal.

In embodiments of the first aspect of the invention, the sensitive cell may be arranged at the first end of the housing, while the inlet is arranged at the second end of the housing.

A second aspect of the invention relates to a measuring method for measuring at least one physiological parameter of a patient. According to the invention, the method comprises:
a connection step of connecting the measuring chamber to the physiological liquid of a patient, the measuring chamber being filled with gas and comprising a sensitive cell making it possible to measure at least one physiological parameter,
a generation step of generating a first interface between the gas present in the measuring chamber and the physiological liquid coming from the patient, the gas being interposed between the physiological liquid and the sensitive cell in such a way as to form a second interface between the gas and the sensitive cell,
a measurement step of measuring at least a first physiological parameter, the measurement being carried out by the sensitive cell through the gas interposed between the physiological liquid and the sensitive cell.

The measuring method follows the same technical principle as the measuring device; it makes it possible to generate a liquid/gas interface at which the physiological liquid comes into contact with the gas contained in the measuring chamber. The physiological liquid then transmits the pressure of the CSF to the gas, which itself transmits this pressure to the sensitive cell of the measuring sensor.

In embodiments of the second aspect of the invention, the volume of gas interposed between the sensitive cell and the physiological liquid of the patient may be non-zero and less than or equal to 1 ml, in particular said volume of gas may be strictly less than 1 ml, preferably said volume of gas may be less than 0.7 ml. This volume of gas interposed between the sensitive cell and the physiological liquid of the patient makes it possible to control the damping of the pulsatility of the pressure of the physiological liquid. This is so that the damping of pulsatility is acceptable in clinical practice.

In embodiments of the second aspect of the invention, the measuring chamber may be connected to a device for sampling physiological liquid which may consist of the patient's cerebrospinal fluid. In particular, the connection device may consist of a lumbar puncture needle. Preferably, the lumbar puncture needle is connected directly to the measuring chamber.

In embodiments of the second aspect of the invention, the first physiological parameter may correspond to a steady-state pressure or to a dynamic pressure of the physiological liquid of the patient.

In embodiments of the second aspect of the invention, the measuring method may comprise a determination step of determining at least a second physiological parameter through the measurement of the first physiological parameter.

### DESCRIPTION OF THE DRAWINGS

Further features and advantages of the invention will become clear upon reading the description which follows. This is purely illustrative and must be read in conjunction with the attached drawings in which:

FIG. 1 schematically depicts a longitudinal section through a measuring device according to the invention, the measuring device being connected to a lumbar puncture needle.

FIG. 2 schematically depicts the measuring device of FIG. 1 and its medical-technical interactions.

FIG. 3 schematically depicts a measuring device according to the invention.

FIG. 4 is a flowchart schematically illustrating a measuring method according to the invention.

FIG. 5 schematically depicts an experimental setup for conducting a comparative study of hydrostatic pressure measurements taken by a liquid interface sensor and a liquid/air interface sensor.

FIG. 6 depicts a graphical recording of the hydrostatic pressure signals measured during experiment 1, the signals being measured by a liquid interface sensor and a liquid/air interface sensor according to the setup of FIG. 5.

FIG. 7 is a close-up of the recording of FIG. 6.

FIG. 8 depicts a graphical recording of the hydrostatic pressure signals measured during experiment 2 in which the signals are measured by a liquid interface sensor and a liquid/air interface sensor according to the setup of FIG. 5.

FIG. 9 is a close-up of cycle C8 of the graph of the recording of FIG. 8.

FIG. 10 depicts a Bland-Altman plot constructed with data from experiment 2.

FIG. 11 depicts a graphical recording of the pressure signals measured during an experiment 3 in which the pressure is pulsatile, the signals being measured by a liquid interface sensor and a liquid/air interface sensor according to the setup of FIG. 5.

FIG. 12 is a close-up of cycle C1 of the graph of the recording of FIG. 11.

FIG. 13 is a close-up of cycle C7 of the graph of the recording of FIG. 11.

FIG. 14 depicts a results diagram of an experiment 4 conducted using the setup of FIG. 5, the diagram depicting the evolution of the pulsatility of the pressure measured by the liquid/air interface sensor and the liquid interface sensor as a function of the volume of air of the liquid/air interface.

FIG. 15 depicts a diagram illustrating a constitutive relation of the damping of the pulsatility of the pressure measured by the liquid/air interface sensor as a function of the volume of air of the liquid/air interface.

### DESCRIPTION OF THE EMBODIMENTS

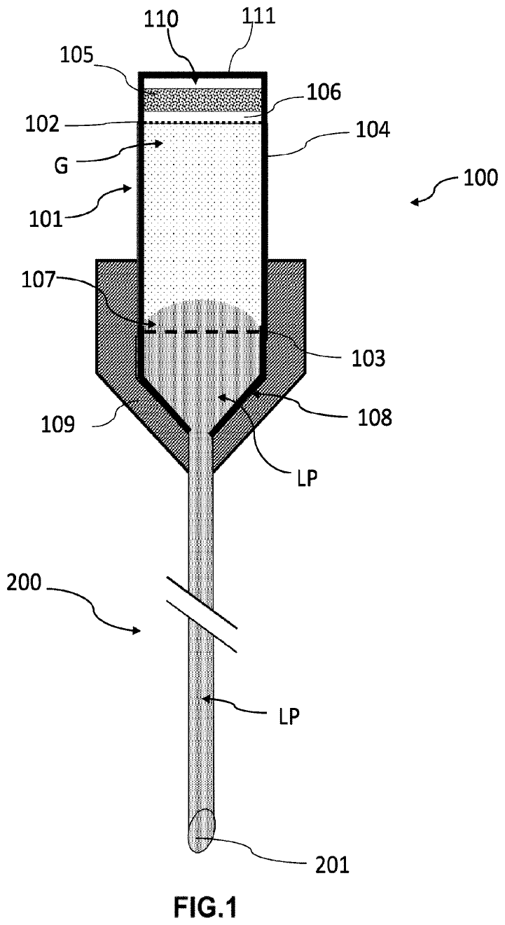

With reference to FIGS. 1 to 3, the invention relates to a measuring device 100 for measuring at least one physiological parameter. More particularly, the measuring device 100 is configured to measure the pressure of the physiological liquid drawn off from the body of a patient. For example, the invention relates to a measuring device 100 for measuring CSF pressure, which is an indirect measurement of a patient's intracranial pressure.

As shown in FIGS. 1 to 3, the measuring device 100 comprises a housing 101. The housing 101 defines the outer casing of the measuring device 100. The housing 101 extends longitudinally between a first end 102 and a second end 103 opposite the first end 102. The housing 101 comprises a measuring chamber 104. The measuring chamber 104 extends inside the housing 101. In this example, the measuring chamber 104 extends between the first end 102 and the second end 103 of the housing 101. The measuring chamber 104 is filled by default with a gas G. For example, the measuring chamber 104 may be filled with air. However, it is also possible to fill the measuring chamber 104 with another gas.

In this example, the measuring chamber 104 has a non-zero volume of less than or equal to 1 ml. In particular, the internal volume of the measuring chamber 104 may be less than 1 ml. Preferably, the internal volume of measuring chamber 104 is less than 0.7 ml.

According to an embodiment of the invention, the internal volume of the measuring chamber 104 may be between 0.2 ml and 0.5 ml. However, the internal volume may also be between 0.3 ml and 0.4 ml.

In this case, the internal volume of the measuring chamber 104 is cylindrical. The internal volume is delimited by the internal walls of the housing 101.

As shown in FIGS. 1 to 3, the housing 101 comprises a sensitive cell 105 making it possible to measure at least one physiological parameter. The sensitive cell 105 is arranged in contact with the gas G contained in the internal volume of the measuring chamber 104. In this example, the sensitive cell 105 is arranged at the first end 102 of the housing 101. In particular, the sensitive cell 105 could be of piezoelectric type. A piezoelectric sensitive cell can convert a pressure measurement into an electrical signal.

As shown in FIGS. 1 to 3, the housing 101 comprises a sealing membrane 106. The sealing membrane 106 is interposed between the sensitive cell 105 and the gas G contained in the internal volume of the measuring chamber 104. In this example, the sealing membrane 106 is arranged at the first end 102 of the housing 101. The sealing membrane 106 isolates the sensitive cell 105. The sealing membrane 106 improves the safety of the device, keeping the electronic components isolated from the internal volume of the measuring chamber 104. The sealing membrane may be made of an air- and water-impermeable medical polymer such as silicone or polyurethane.

In the example of FIGS. 1 to 3, the housing 101 comprises an inlet 107 for the intake of a physiological liquid LP of a patient. Preferably, the housing 101 has only one inlet 107. This is because, unlike the prior art, it is not sought to discharge the gas present in the measuring chamber 104. Conventionally in clinical practice, contact between air and a physiological fluid is avoided, and in fact the injection of a small volume of air into the blood system runs the risk of an embolism which may have serious consequences. However, injecting the same small volume of air into the nervous system will have no effect. Therefore, a single inlet 107 makes it possible to control the formation of a liquid/gas interface within the measuring chamber 104. The inlet 107 is arranged such that the physiological liquid LP comes into contact with the gas G. The gas G is then interposed between the sensitive cell 105 and the physiological liquid LP of the patient. For this purpose, the inlet 107 may be arranged on the housing 101 at the opposite end to the sensitive cell 105. In the example of FIGS. 1 to 3, the inlet 107 is arranged at the second end 103 of the housing 101 while the sensitive cell 105 is arranged at the first end 102 of the housing 101. In this case, the inlet 107 is constituted by an opening in the housing 101, the outline of the inlet 107 being shown in dotted lines in FIGS. 1 to 3. However, other configurations and other housing shapes are conceivable to allow the gas G to be interposed between the physiological liquid LP and the sensitive cell 105.

As shown in FIGS. 1 to 3, the measuring device 100 comprises a connector 108. The connector 108 is arranged at the inlet 107 of the housing 101. In this example, the connector 108 sealingly connects the measuring chamber 104 to a connection device 200 for the patient's physiological liquid LP. In this example, the connector 108 is in the form of a male conical portion that is configured to connect with a female conical endpiece 109 of the connection device 200.

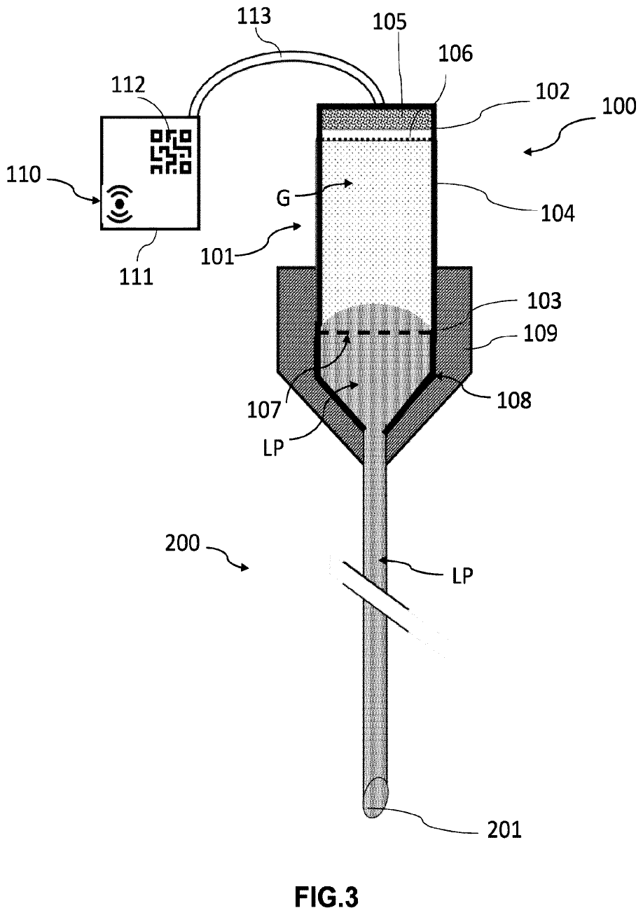

In the example of FIGS. 1 and 3, the female conical endpiece 109 of the connection device 200 connects directly to the conical connector 108 of the housing 101. As shown in FIGS. 1 and 3, the connection device 200 comprises a lumbar puncture needle 201.

Preferably, the connection device 200 consists of a lumbar puncture needle 201. According to this configuration, the measuring device 100 is directly connected to the lumbar puncture needle 201. This makes it easier for practitioners to use the measuring device 100.

Alternatively, the connector 108 may be connected via a three-way stopcock to the conical endpiece 109 of the lumbar puncture needle 201.

According to another alternative (not shown) for the use of the invention, the connector 108 may be connected to tubing interposed between the measuring device 100 and the lumbar puncture needle 201.

Lumbar puncture consists in inserting a needle through the patient's skin into the lumbar sac in order to gain access to the CSF. Typically, a lumbar puncture may be performed to collect CSF so that it can be submitted for biochemical or biological analysis. Lumbar puncture is thus commonly used in cases of suspected meningitis, or inflammatory or neurodegenerative neurological disease.

In addition to the window for biochemical analysis opened on the nervous system, lumbar puncture also offers a window for biomechanical analysis of the nervous system through measurement of the pressure of the CSF. Lumbar puncture is currently the least traumatic technique for accessing the cerebrospinal fluid. To be specific, in the context of the present invention, lumbar puncture constitutes a preferred means of accessing the CSF which constitutes the physiological liquid LP within the meaning of the invention. It is then possible to measure, through the pressure of the CSF, the intracranial pressure. However, the measuring device 100 may also operate with other CSF access methods such as an intraventricular drain.

According to an embodiment of the invention, the inlet 107 may be openable and closable. For this purpose, the inlet 107 may be connected via the connector 108 to an at least two-way stopcock.

As shown in FIGS. 1 to 3, the measuring chamber 104 according to the invention makes it possible to generate a first interface between the gas G which is contained in the internal volume of the measuring chamber 104, and the physiological liquid LP of the patient which enters the measuring chamber 104 via the inlet 107. When the physiological liquid LP enters the measuring chamber 104, the gas G is interposed between the sensitive cell 105 and the physiological liquid LP of the patient, which generates a second interface between the gas G and the sensitive cell 105. The liquid/gas interface and the gas/sensitive cell 105 interface are formed by virtue of the conformation of the measuring chamber 104. However, the arrangement of the sensitive cell 105 relative to the inlet 107 may also contribute to generating said interfaces. In the example illustrated in FIGS. 1 to 3, the liquid/gas interface has a meniscal conformation, this is due in part to the cylindrical shape of the measuring chamber 104. However, the physicochemical properties of the gas G and of the physiological liquid LP also contribute to establishing a particular shape of the meniscus at the measurement interface.

As shown in FIGS. 1 to 3, the measuring device 100 comprises transmission means 110. The transmission means 110 transmit the measurements of the physiological parameter to a digital terminal 300 remote from the measuring device 100.

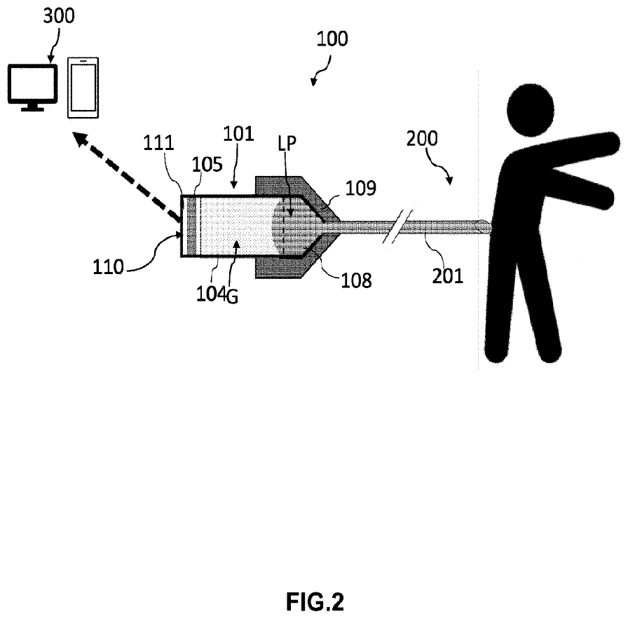

In the example of FIGS. 1 and 2, the transmission means 110 are arranged in an electronic compartment 111 arranged in the extension of the first end 102 of the housing 101. The electronic compartment 111 includes an electronic board 112 connected, on the one hand, to the transmission means 110, and on the other hand, to the sensitive cell 105. The data measured by the sensitive cell 105 are transmitted to the transmission means 110 which relay them to the remote digital terminal 300. In the case of a piezoelectric sensitive cell of a pressure sensor, the data measured is transmitted to the transmission means in the form of an electrical signal.

In the example of FIGS. 1 to 3, the transmission means 110 are wireless. For example, the transmission means 110 may consist of a transmitter/receiver of electromagnetic waves such as radio waves, Bluetooth, WIFI, etc. Under certain specific conditions, the transmission means 110 may be wired.

The electronic compartment 111 may also include a battery which supplies power to the electronic board 112 and the electronic components to which the electronic board 112 is connected. The battery may be rechargeable or single-use.

In the example of FIG. 3, the electronic compartment 111 is separate from the housing 101. In this configuration, the measuring device 100 comprises an external electric cable 113 which detachably connects the sensitive cell 105 to the electronic board 112. For reasons of hygiene, the housing 101 is a single-use housing, and thus an electronic compartment 111 separate from the housing 101 allows the electronic compartment 111 to be used for several patients.

The digital terminal 300 may be a smartphone, a tablet, a computer or any other device making it possible to receive data and analyze same using an algorithm stored in its memory or a remote server accessible via a telecommunications network.

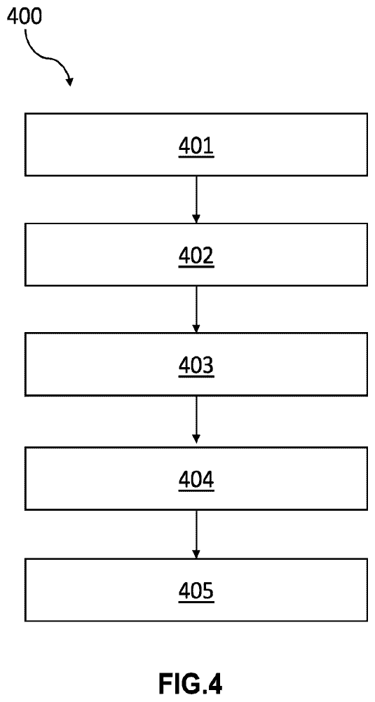

As shown in FIG. 4, the invention also relates to a measuring method 400 for measuring at least one physiological parameter of a patient.

The measuring method 400 comprises a connection step 401 of connecting a measuring chamber to the physiological liquid of a patient. For example, the measuring chamber may be connected to the patient's physiological liquid through a connection device 200. The connection device 200 may include a lumbar puncture needle. This allows access to the cerebrospinal fluid in order to measure intracranial pressure.

In the example illustrated in FIG. 2, the lumbar puncture needle 201, which is connected to the patient, may also be connected to tubing using a three-way stopcock, for injection in order to carry out dynamic tests.

According to the invention, during the connection step 401, the measuring chamber is filled by default with a gas G and comprises a sensitive cell making it possible to measure at least one physiological parameter. For example, the housing comprises a piezoelectric sensitive cell so as to measure CSF pressure.

As shown in FIG. 4, the measuring method 400 comprises a generation step 402 of generating a first interface between the gas present in the measuring chamber and the physiological liquid coming from the patient. As shown in FIGS. 1 to 3, the gas is interposed between the patient's physiological liquid and the sensitive cell in such a way as to form a second interface between the gas and the sensitive cell. For this purpose, the measuring chamber may be fluidically connected directly to a connection device 200 consisting of a lumbar puncture needle 201.

The measuring chamber may be connected to the lumbar puncture needle 201 and to tubing to perform dynamic tests.

In this example, the gas interposed between the sensitive cell 105 and the patient's physiological liquid has a non-zero volume of less than or equal to 1 ml. The volume of gas interposed between the physiological liquid and the sensitive cell may also be less than 1 ml. In particular, the volume of gas interposed between the physiological liquid and the sensitive cell 105 may be less than 0.7 ml.

According to an embodiment of the invention, the volume of gas interposed between the physiological liquid and the sensitive cell may be between 0.2 ml and 0.5 ml. However, the volume of gas interposed between the physiological liquid and the sensitive cell 105 may also be between 0.3 ml and 0.4 ml.

Thus, the sensitive cell 105 carries out a measurement through a hydropneumatic interface. The hydropneumatic interface has the advantage of damping the physical acceleration experienced by the sensitive cell 105 such as patient movements or vibration induced by the medical environment (monitoring equipment, etc.), while allowing more reliable measurement of the pressure or pulsatility of the CSF.

As shown in FIG. 4, the measuring method 400 comprises a measurement step 403 of measuring at least a first physiological parameter. The measurement of the first physiological parameter is carried out using a sensitive cell while the gas G contained in the measuring chamber is interposed between the physiological liquid and the sensitive cell. For example, CSF pressure is a physiological parameter that can be measured by the measuring method 400, in this case the CSF pressure may constitute the first physiological parameter. A piezoelectric sensitive cell may be used to measure the steady-state hydrostatic pressure or the pulsatile pressure of the CSF.

In the example of FIG. 4, the measuring method 400 may comprise a determination step 404 of determining at least a second physiological parameter through the measurement of the first physiological parameter.

According to a first example, a mean value of the patient's intracranial pressure may be determined. The mean value of intracranial pressure then constitutes the second physiological parameter.

According to a second example, the pulsatility of the patient's intracranial pressure may be determined. The value of intracranial pulsatility then constitutes the third physiological parameter.

Within the framework of a hydrodynamic approach, the method according to the invention makes it possible to determine other physiological parameters such as the frequency of the respiratory cycle, cerebral compliance or resistance to CSF flow.

As shown in FIGS. 3 and 4, the measuring method 400 may comprise a transmission step 405 of transmitting the data measured by the sensitive cell to a digital terminal 300 remote from the sensitive cell. The transmission step 405 may be carried out according to a Bluetooth or WIFI type protocol or indeed in an analog manner via a wired connection.

Upon receipt of the measured data, the digital terminal 300 may execute step 404 and process the signal in order to extract at least one physiological parameter. The patient's intracranial pressure is a parameter that may be determined directly by measuring CSF pressure.

According to the invention, the measuring device 100 is particularly suitable for implementing the measuring method 400.

The measuring device 100 and the measuring method 400 make it possible to reliably and accurately measure CSF pressure in both a hydrostatic approach and a hydrodynamic approach. The hydrostatic approach consists in measuring the steady-state pressure or the pulsatile pressure of the CSF secondary to cyclic cardiac and respiratory oscillations. The hydrodynamic approach consists in applying a known stress to the intracranial fluid system, called a perfusion test, and analyzing the system's response to this stress.

To explore the hydrostatic component of the pressure of the CSF, the measuring chamber 104 is connected to a connection device 200 which is in fluidic connection with the nervous system, for example, via a lumbar puncture. Variations in the electrical signal measured by the sensitive cell 105 reflect the variations in CSF pressure.

To explore the hydrodynamic component of the pressure of the CSF, fluidic stress is applied to the CSF by injecting physiological serum at a constant flow rate through an inlet port at the lumbar puncture site. This perfusion test is carried out via an inlet port created in parallel with the first port to which the connection device 200 is connected and which places the measuring device 100 in fluidic contact with the CSF.

### EXPERIMENTAL RESULTS

In order to address the technical problems set out in this document, the inventors conducted a comparative experimental study between the pressure measurements obtained by a sensor with a liquid/sensitive cell interface, referred to as a “fluid sensor” 501, and the pressure measurements obtained by a sensor with two liquid/gas/sensitive cell interfaces, referred to as a “hydropneumatic sensor” 502. This work was carried out within the same experimental setup 500 which uses a physiological serum S instead of the patient's physiological liquid.

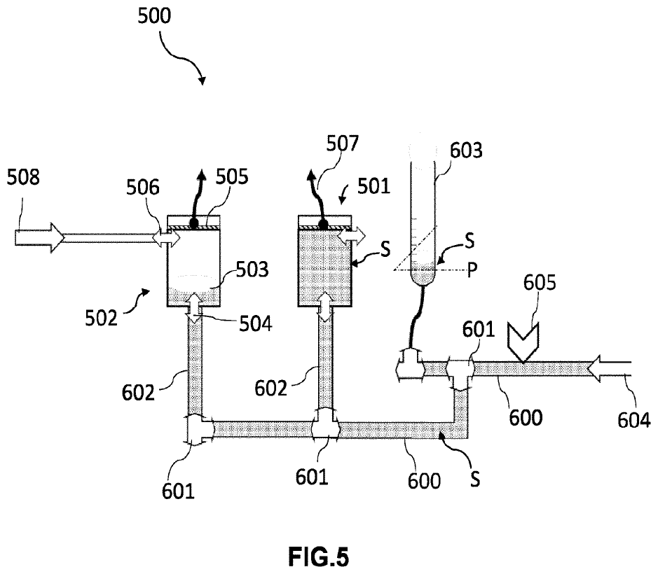

FIG. 5 schematically depicts the experimental setup 500. The two sensors 501, 502 are of piezoelectric type, the brand “Transpac™” marketed by the company ICUMED, for the measurement of invasive blood pressure. This pressure measuring device comprises a measuring chamber 503 the internal volume of which is 0.2 ml. Furthermore, this measuring device comprises a fluid inlet 504 which is connected to tubing 600 filled with physiological serum S. The fluid inlet 504 is equipped with a three-way stopcock 504 to control the connection or disconnection of the sensor with the physiological serum S. The measuring device used comprises, opposite the inlet 504, an end wall composed of the sensitive cell of the piezoelectric sensor 505. In the vicinity of the piezoelectric sensor 505, the side wall of the measuring chamber 503 comprises a purge opening 506, the opening and closing of which are controlled by a manual valve or stopcock. The piezoelectric sensor 505 of this measuring device has a sensitivity of plus or minus one percent and a linearity of 1%. Lastly, this piezoelectric sensor is designed to measure changes in pressure between −20 mmHg and 300 mmHg.

In the experimental setup 500 shown in FIG. 5, the two sensors 501 and 502 are mounted on a support and held horizontally in the same plane. In this case, the two sensors 501, 502 are connected to tubing 600 in parallel. The tubing 600 includes a three-way stopcock 601 at each junction point with a connecting tube 602 connecting the tubing 600 to the sensors 501 and 502.

The sensitive cell of each piezoelectric sensor 501 and 502 is connected by an electric cable 507 independently to an amplifier then a converter before being recorded on a computer using the software “ICM+®”.

In the experimental setup 500 of FIG. 5, the purge opening 506 of the hydropneumatic sensor 502 is connected to a syringe 508 containing air. The syringe 508 is graduated and makes it possible to control the volume of air in the measuring chamber 503, that is to say, the volume of air interposed between the sensitive cell of the piezoelectric sensor 505 and the physiological serum S.

The experimental setup 500 also includes a graduated column 603 which is held vertically. The graduated column 603 comprises a fluidic connection at its lower end. The graduated column 603 allows the inventors to visualize the pressure applied to the sensors 501, 502 by measuring the water column within the graduated column 603. For this purpose, the sensors 501, 502 are positioned such that their piezoelectric sensitive cells 505 are located in a horizontal plane P passing through the zero level defined on the graduated column. In the example of FIG. 5, the graduated column 603 is arranged at one end of a first branch of the tubing 600. The second branch of the tubing 600 is mounted in parallel with the first branch and supplies the sensors 501, 502 through connection stopcocks 601 which are arranged at each junction point with a connection tube 602.

The tubing 600 filled with physiological serum S is connected to an electric syringe pump 604. This syringe pump 604 is configured to apply static pressures in successive stages between −20 mmHg and 50 mmHg. These values correspond to the CSF pressures that may be measured in a patient both in physiology and in pathology. The electric syringe pump 604 used in this experiment is a FRESENIUS® syringe pump.

The connection stopcock 601 makes it possible to connect, where applicable with the tubing 600, an oscillatory generator 605 in order to create dynamic wave pressures with a frequency of 1 Hz and amplitudes adjustable between 0.1 and 10 mmHg. These amplitude values correspond to those of the intracranial pressure in a patient both in physiology and in pathology. The oscillatory pressure generator 605 used in this experiment is of RADOMEDIC® type.

Note that when delivered, the sensors 501 and 502 naturally contain air in their measuring chambers 503.

For the fluid sensor 501, an air purge is carried out through the opening 506 of the measuring chamber 503 and by filling it with physiological serum. Thus, the sensitive cell is in direct contact with the fluid in the measuring chamber. This air purge protocol is the operating procedure usually recommended for measuring fluid pressure with this type of sensor in clinical practice, especially for invasive measurement of arterial or venous blood pressure.

Once the fluid sensor 501 is purged of air, it is in fluidic connection with the physiological serum S.

For the hydropneumatic sensor 502, no air purge is performed. As the measuring chamber 503 has a volume of 0.2 ml, it will therefore contain 0.2 ml of air. This volume of air will therefore be interposed between the sensitive cell 505 and the inlet 504. The hydropneumatic sensor will then be connected to the physiological serum by the connection 602. A controlled volume of air will thus be added between the piezoelectric sensor 505 and the physiological serum S. The inventors thus generate a liquid/gas measurement interface within the measuring chamber in accordance with the present invention, corresponding to the hydropneumatic principle.

The two sensors, fluid 501 and hydropneumatic 502, are in fluidic connection, by virtue of the physiological serum S, with the graduated column 603, with the electric syringe pump 604 and with the dynamic pressure generator 605.

### Experiment 1

Experiment 1 consists in recording, using the software “ICM+®”, the pressure measurements taken by the fluid sensor 501 and the pressure measurements taken by the hydropneumatic sensor 502 simultaneously. As a reminder, the volume of air in the hydropneumatic sensor 502 is that of the internal volume of the measuring chamber 503, i.e. 0.2 ml.

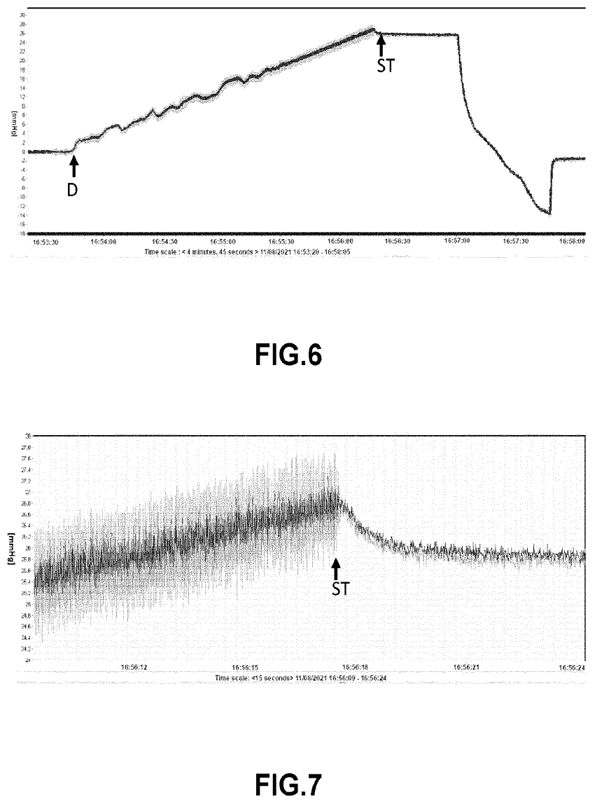

Measurements of steady-state hydrostatic pressure were acquired at 100 Hz for a recording duration of 5 minutes. By injecting physiological serum, the hydrostatic pressure could be gradually increased in the two sensors 501 and 502. FIG. 6 corresponds to the recording of this experiment. In this figure, the gray signal corresponds to the fluid sensor 501 and the black signal corresponds to the hydropneumatic sensor 502.

In experiment 1, the electric syringe pump 604 rests on the tabletop on which the experimental setup 500 is placed. In the baseline state, the values measured by the fluid sensor 501 and by the hydropneumatic sensor 502 are identical: the two lines representing the two sensors are superimposed in FIG. 6. Arrow D indicates the switching on of the electric syringe pump 604 at time 16 h 53 min and 30 sec. Upon switching on, a progressive increase in pressure is observed from 0 mmHg up to approximately 25 mmHg at the recording time of 16 h 56 min and 18 sec, when the electric syringe pump 604 is stopped (arrow ST).

The electric cables of the sensors 501 and 502 are connected separately to the computer running the “ICM+” software; consequently the inventors were able to study and compare the signals from the fluid sensor 501 and those from the hydropneumatic sensor 502. This is also the case in the subsequent experiments.

In the recording of FIG. 6, the inventors noted that the signals of the two sensors 501, 502 were superimposed until the switch-on D of the electric syringe pump 604. With the two signals superimposed, only the black signal is visible. When the electric syringe pump is switched on at D, there is on the one hand an increase in the hydrostatic pressure of the two signals following a similar curve, and on the other hand an oscillation of the gray signal recorded by the fluid sensor 501. FIG. 7 is a close-up of FIG. 6, and shows that the signal from the hydropneumatic sensor 502 (in black) also exhibits an oscillation, however its amplitude is much less than that of the fluid sensor 501 (in gray). As soon as the electric syringe pump 604 stops ST, the oscillation disappears; the two signals from the two sensors, fluid 501 and hydropneumatic 502, are then superimposable.

This oscillation is not induced by the pulse generator 607, which is switched off. This oscillation appeared as soon as the electric syringe pump 604 was switched on, and stopped as soon as it was switched off. The inventors thus concluded that the mechanical vibration of the electric syringe pump 604 resting on the table is transmitted to the fluid sensor 501, whereas the hydropneumatic sensor 502 dampens this vibration. The density of air is 1.29 kg/m³ while the density of a liquid like water is 1000 kg/m³. Water therefore has a density approximately 1000 times greater than that of air. The 0.2 ml measuring chamber of the fluid sensor therefore has a mass of ≈0.2 g, whereas the 0.2 ml measuring chamber of the hydropneumatic sensor only has a mass of ≈0.26 μg. During the acceleration of the fluid sensor induced by a vibration wave, the mass of water in the measuring chamber (≈0.2 g) creates a mechanical stress on the sensitive cell 505 which results in a measurement artifact, whereas in the measuring chamber of a hydropneumatic sensor the low mass of the air (≈0.26 μg) creates only a very low mechanical stress on the sensitive cell, thus damping the vibration wave.

This experiment shows that the hydropneumatic sensor 502 dampens the physical acceleration transmitted during vibration to the measuring device. From a practical point of view, this translates into an improvement in the accuracy of the CSF pressure measurement.

During this experiment 1, we also compared the two sensors from an arithmetic point of view. The mean value±standard deviation of the absolute difference between the fluid sensor and the hydropneumatic sensor is 0.19±0.21 mmHg, and the 95% confidence interval of the absolute difference is 0.41 mmHg. That is, 95% of the difference values between the fluid sensor and the hydropneumatic sensor are within the range of ±0.41 mmHg.

During experiments 2, 3 and 4, the electric syringe pump 604 is moved away from the manipulation table so that the physical vibration it generates is not transmitted directly to the sensors and does not create an artifact.

### Experiment 2

Experiment 2 uses the setup 500 to study the impact of the volume of air in the measuring chamber on the accuracy of the static pressure measurement of the hydropneumatic sensor.

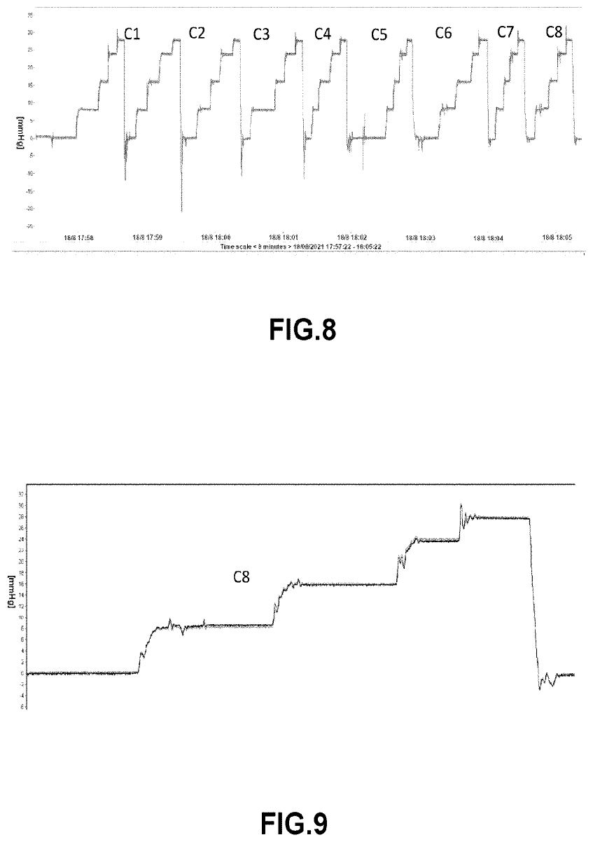

The pressure of the physiological serum is increased in successive static stages of around ten seconds followed by a return to zero. The signals from the fluid 501 and hydropneumatic 502 sensors are recorded simultaneously at 100 Hz. Several pressure increase cycles are carried out with different volumes of air in the measuring chamber of the hydropneumatic sensor by adding air with the syringe 508, at ambient temperature and atmospheric pressure. FIG. 8 reproduces the recording which lasts 23 minutes. The first pressure increase cycle C0 takes place without the addition of air; the volume of air in the measuring chamber is therefore 0.2 ml. The two curves are perfectly superimposable. Then 0.1 ml of air is added to the measuring chamber of the hydropneumatic sensor. Cycle C1 corresponds to the different pressure stages with an addition of 0.1 ml of air. The two curves are perfectly superimposable. Cycles C0 to C8 are performed with increasing volumes of air, described in table 1.

TABLE 1

| Cycle | C0 | C1 | C2 | C3 | C4 | C5 | C6 | C7 | C8 |
|---|---|---|---|---|---|---|---|---|---|
| Volume of air added (ml) | 0 | 0.1 | 0.5 | 1 | 2 | 6 | 12 | 36 | 64 |

Looking at the curve in the various cycles, it can be seen that whatever the volume of air interposed between the sensitive cell of the piezoelectric sensor 505 and the physiological serum S, the signals measured by the fluid sensor 501 and by the hydropneumatic sensor 502 are superimposed.

FIG. 9 is a close-up of the graph of cycle C8 of the experiment, that is, the cycle for which a volume of 64 ml of air is interposed between the sensitive cell of the piezoelectric sensor 505 and the physiological serum S. The volume of air of 64 ml is the maximum in the context of experiment 2. The two signals from the fluid sensor 501 (in gray) and from the hydropneumatic sensor 502 (in black) can be distinguished in this graph. The two signals are fully superimposed throughout most of the recording.

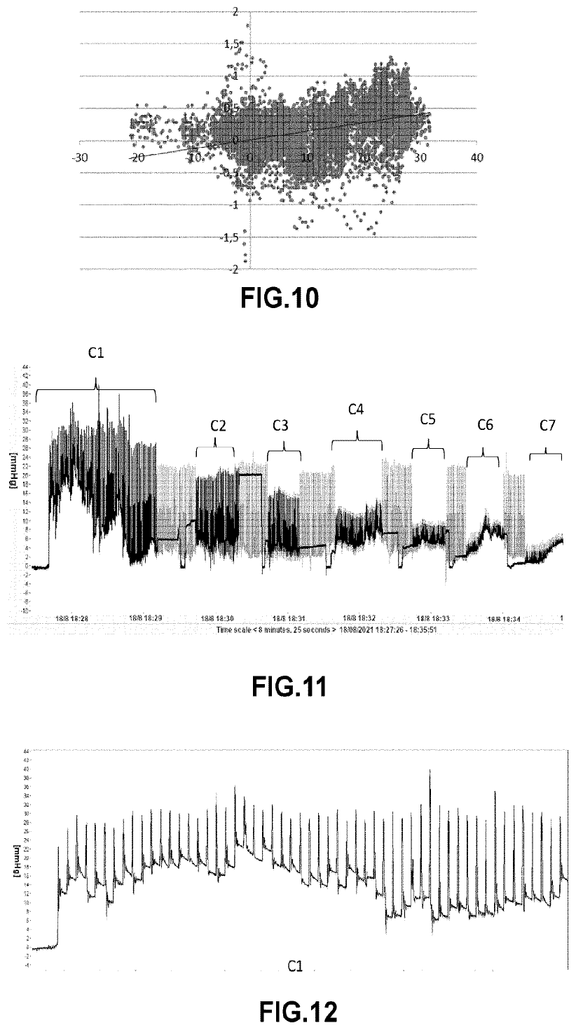

FIG. 10 is a Bland-Altman plot produced from the experimental data from experiment 2. The X axis of the graph corresponds to the mean value of the fluid 501 and hydropneumatic 502 pressure measurements expressed in mmHg. The Y axis of the graph corresponds to the difference between the fluid 501 and hydropneumatic 502 pressure measurements expressed in mmHg. A linear regression is then performed.

The mean value±standard deviation of the absolute difference between the fluid sensor and the hydropneumatic sensor is 0.24 mmHg±0.17 mmHg, and the 95% confidence interval of the absolute difference is 0.33 mmHg. That is, 95% of the difference values between the fluid sensor and the hydropneumatic sensor are within the range of ±0.33 mmHg.

In experiment 2, the pressure varied in values between −10 and +30 mmHg, which corresponds to the values found in clinical practice. 0.33 mmHg represents at most ≈1% of the pressure measurement value. This variation in measurement is acceptable in clinical practice. It should also be noted that 1% also corresponds to the sensitivity of the sensor.

The scientific literature specifies that a blood pressure sensor is reliable when it offers a percentage error of 5% of the measurement compared to the patient's actual blood pressure.

Experiment 2 shows that a volume of air of between 0.2 ml and 64 ml interposed in the measuring chamber of a hydropneumatic sensor 502 allows a reliable measurement of the pressure, in comparison with that of a fluid sensor 501.

### Experiment 3

Experiment 3 uses the setup 500 to study the impact of the volume of air in the measuring chamber on the accuracy of the dynamic pressure measurement of the hydropneumatic sensor 502.

For this purpose, the oscillatory generator 605 makes it possible to create dynamic wave pressures with a frequency of 1 Hz and amplitudes adjustable between 0.1 and 10 mmHg. The signals from the fluid 501 and hydropneumatic 502 sensors are recorded simultaneously at 100 Hz. The recording duration is 8 minutes.

For this experiment, the static pressure is stable, with no variation in the mean pressure. Only the dynamic pressure is modified by virtue of the oscillatory generator 605, observing the impact of the volume of air on the pulsatility of the pressure measured by the hydropneumatic sensor 502. Multiple recordings are made at different pulsatility levels. For each pulsatility level, the volume of air in the measuring chamber of the hydropneumatic sensor is gradually increased by adding air using the syringe 508. FIG. 11 reproduces one of the recordings. The black line corresponds to the hydropneumatic sensor 502 showing the damping upon each cycle of increasing the volume of air. Conversely, the gray line corresponding to the fluid sensor 501 remains unchanged from one cycle to another. Cycles C1 to C7 are performed with increasing volumes of air, described in table 2.

TABLE 2

| Cycle | C1 | C2 | C3 | C4 | C5 | C6 | C7 |
|---|---|---|---|---|---|---|---|
| Volume of air added (ml) | 0.2 | 0.5 | 1 | 2 | 5 | 10 | 20 |

Each time air is added to the measuring chamber, the pulsatility of the signal measured by the hydropneumatic sensor 502 decreases proportionally with damping.

FIG. 12 is a close-up of cycle C1. In this recording, it is not possible to distinguish the gray signal from the fluid sensor 501, which is superimposed on the black signal from the hydropneumatic sensor 502. With a very small volume of air, signal damping is minimal.

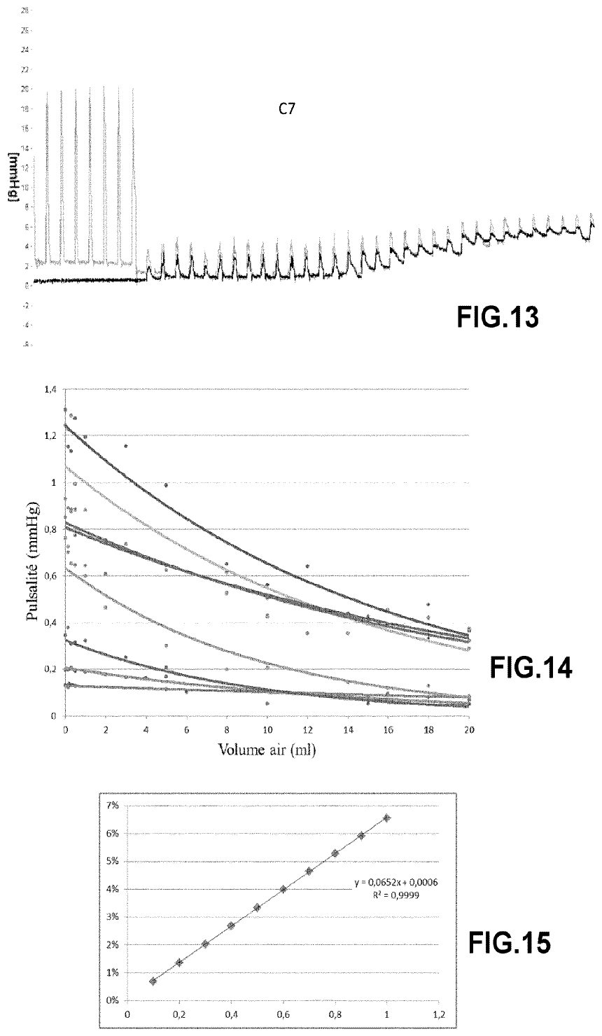

FIG. 13 is a close-up of cycle C7. At the beginning of this recording, the gray signal from the fluid sensor 501 shows the pulsatility produced by the wave generator 605. As soon as the hydropneumatic sensor is connected with 20 ml of air, there is immediate damping of the gray signal from the fluid sensor (502) synchronous with the black signal from the hydropneumatic sensor 502, these two sensors being connected in parallel.

Experiment 3 shows that in the case of pulsatile pressure, the volume of air of the hydropneumatic sensor 502 modifies the dynamic measurement of the pressure and consequently influences the accuracy of the measurement.

### Experiment 4

Experiment 4 aims to determine the volume of air in the measuring chamber of the hydropneumatic sensor 502 in order to provide an accurate and clinically acceptable measurement of pulsatility.

Experiment 4 consists in carrying out a series of eight manipulations at different pulsatility levels during which the volume of air in the hydropneumatic sensor 502 is gradually increased by adding air at ambient temperature and atmospheric pressure using the syringe 508. The experiment is carried out over a period of 10 minutes with a pulsatility of 1 Hz produced by the generator 605, the oscillation of which is adjustable. The recording is carried out using the software “ICM+R” and an acquisition at 100 Hz. For each volume of air (cycles C1 to C7), the pulsatility is not calculated by the difference between maximum and minimum, but with a Fourier transform by measuring the power of the fundamental harmonic in the frequency range of the signal pulse. This gives a numerical value of the pulsatility for each volume of air from 0.2 ml to 20 ml. A graphical representation is obtained using the software XL characterizing the damping of the pulsatility. An exponential regression analysis is performed, obtaining its mathematical equation and the Pearson coefficient. For each oscillatory condition, it is thus possible to obtain an exponential curve describing the constitutive relation of the damping of pulsatility as a function of the volume of air.

FIG. 14 illustrates the eight experiments. Each point corresponds to a pulsatility value for different volumes of air of the hydropneumatic sensor 502. Each curve corresponds to the evolution of the pulsatility value measured during the different manipulations. Transferring the data to this one graph allowed the inventors to plot the exponential regression curves of each manipulation.

From the exponential regression equations of the data from the eight manipulations of experiment 4, the inventors calculated the theoretical variation in pulsatility damping (Δpulsatility) induced for each addition of an elementary volume of 0.1 ml of air at the hydropneumatic sensor 502. The results are expressed as the percentage of pulsatility damping per elementary volume of air. These results make it possible to establish the mean values and the standard deviation of the pulsatility damping of the hydropneumatic sensor 502 as a function of the volume of air in the measuring chamber. The results are given in table 3 below.

TABLE 3

| | | | Volume air (ml) | | | | | | | | | |
|---|---|---|---|---|---|---|---|---|---|---|---|---|
| | Equation | | 0.1 | 0.2 | 0.3 | 0.4 | 0.5 | 0.6 | 0.7 | 0.8 | 0.9 | 1 |
| 16 | y = 0.1292e^{-0.025x} | Δpulsatilité | 0.25% | 0.50% | 0.75% | 1.00% | 1.24% | 1.49% | 1.73% | 1.98% | 2.22% | 2.47% |
| 17 | y = 0.2046e^{-0.067x} | Δpulsatilité | 0.67% | 1.33% | 1.99% | 2.64% | 3.29% | 3.94% | 4.58% | 5.22% | 5.85% | 6.48% |
| 18 | y = 0.323e^{-0.106x} | Δpulsatilité | 1.05% | 2.10% | 3.13% | 4.15% | 5.16% | 6.16% | 7.15% | 8.13% | 9.10% | 10.06% |
| 19 | y = 1.2762e^{-0.073x} | Δpulsatilité | 0.73% | 1.45% | 2.17% | 2.88% | 3.58% | 4.29% | 4.98% | 5.67% | 6.36% | 7.04% |
| 20 | y = 1.0683e^{-0.067x} | Δpulsatilité | 0.67% | 1.33% | 1.99% | 2.64% | 3.29% | 3.94% | 4.58% | 5.22% | 5.85% | 6.48% |
| 21 | y = 0.6533e^{-0.115x} | Δpulsatilité | 1.14% | 2.27% | 3.39% | 4.50% | 5.59% | 6.67% | 7.73% | 8.79% | 9.83% | 10.86% |
| 22 | y = 0.8277e^{-0.048x} | Δpulsatilité | 0.48% | 0.96% | 1.43% | 1.90% | 2.37% | 2.84% | 3.30% | 3.77% | 4.23% | 4.69% |
| 23 | y = 0.807e^{-0.044x} | Δpulsatilité | 0.44% | 0.88% | 1.31% | 1.74% | 2.18% | 2.61% | 3.03% | 3.46% | 3.88% | 4.30% |
| | | moyenne | 0.68% | 1.35% | 2.02% | 2.68% | 3.34% | 3.99% | 4.64% | 5.28% | 5.92% | 6.55% |
| | | ds | 0.30% | 0.60% | 0.89% | 1.18% | 1.47% | 1.75% | 2.03% | 2.30% | 2.57% | 2.84% |

As shown in table 3, the mean pulsatility damping of a hydropneumatic sensor 502 is less than 6.55% for a volume of air of less than 1 ml. Therefore, taking the accuracy of the blood pressure measurement as a reference, the inventors concluded that the volume of air of the hydropneumatic sensor must be less than 0.7 ml to ensure reliable measurements (<5%) of CSF pulsatility.

The inventors determined a constitutive relation making it possible to determine the percentage of damping of the pulsatility ΔP as a function of the volume of air Vair (in ml). This law is expressed according to the following equation:

$$Ap = 0.0652 * Vair + 0.0006$$

This linear law, as shown in FIG. 15, makes it possible to define that a volume of air of strictly less than 0.7 ml makes it possible to measure the pulsatility of the pressure of a physiological liquid such as CSF with a hydropneumatic sensor, with a measurement variability of less than 5%.

According to these experimental results, it is thus possible to determine the volume of the measuring chamber of the measuring device as non-zero and less than 1 ml. In the case of CSF, when establishing the liquid/gas interface, the physiological values that the CSF pressure may take are likely to cause only a slight variation in the volume of gas initially contained at atmospheric pressure in the measuring chamber such that the gas initially contained at atmospheric pressure in the measuring chamber still has a non-zero volume of less than or equal to 1 ml.

This observation applies to the volume of gas interposed between the CSF and the sensitive cell in the context of the measuring method defined according to the invention.

These experimental results show that it is possible to measure the pressure, whether steady-state or pulsatile, of a biological liquid through a hydropneumatic interface. The invention may thus be used to measure other physiological parameters such as bladder pressure, arterial pressure, venous pressure, interstitial tissue pressure in the context of compartment syndrome, ocular pressure, pressure in the upper airways, pressure in the digestive tract and any other biological body fluid pressure. For each of these applications, the biological liquid of interest is placed in contact with the measuring device 100 as described above, in a known manner, the biological liquid of interest may be probed or drawn off, depending on its nature.

1. A measuring device for measuring at least one physiological parameter, the measuring device including a housing that extends longitudinally between a first end and a second end opposite the first end, the housing comprises:
a measuring chamber which is arranged in the housing, the measuring chamber has an internal volume which is filled with gas,
a sensitive cell making it possible to measure at least a first physiological parameter, the sensitive cell being arranged in contact with the gas contained in the internal volume of the measuring chamber, and
an inlet for the intake of a physiological liquid of a patient into the measuring chamber,
wherein the inlet is arranged such that the physiological liquid comes into contact with the gas and the gas is interposed between the sensitive cell and the physiological liquid of the patient, the measuring chamber making it possible to generate two interfaces: a first interface between the gas contained in the internal volume and the physiological liquid of the patient which enters the measuring chamber through the inlet, and a second interface between the gas contained in the measuring chamber and the sensitive cell.

2. The measuring device as claimed in claim 1,
wherein the measuring chamber has an internal volume that is non-zero and less than or equal to 1 ml.

3. The measuring device as claimed in claim 1, wherein the measuring chamber has an internal volume of between 0.2 ml and 0.5 ml.

4. The measuring device as claimed in claim 1, wherein the sensitive cell is a piezoelectric cell of a pressure sensor.

5. The measuring device as claimed in claim 1, wherein the housing comprises a sealing membrane which is interposed between the sensitive cell and the internal volume of the measuring chamber, the sealing membrane protecting the sensitive cell.

6. The measuring device as claimed in claim 1, wherein the internal volume of the measuring chamber is cylindrical.

7. The measuring device as claimed in claim 1, comprising a connector arranged at the inlet of the housing, the connector sealingly connecting the measuring chamber to a connection device for connection to the physiological liquid of the patient.

8. The measuring device as claimed in claim 1, wherein the inlet is openable and closable.

9. The measuring device as claimed in claim 1, comprising transmission means which transmit the measurements of the first physiological parameter to a remote digital terminal.

10. The measuring device as claimed in claim 1, wherein the gas in the measuring chamber includes atmospheric air.

11. The measuring device as claimed in claim 1, wherein the sensitive cell is arranged at the first end of the housing, while the inlet is arranged at the second end of the housing.

12. A measuring method for measuring at least one physiological parameter of a patient, wherein the method comprises:
a connection step of connecting a measuring chamber connection device to the physiological liquid of a patient, the measuring chamber being filled with gas and comprising a sensitive cell making it possible to measure at least one physiological parameter,
a generation step of generating a first interface between the gas present in the measuring chamber and the physiological liquid coming from the patient, the gas being interposed between the physiological liquid and the sensitive cell in such a way as to form a second interface between the gas and the sensitive cell, and
a measurement step of measuring at least a first physiological parameter, the measurement being carried out by the sensitive cell through the gas interposed between the physiological liquid and the sensitive cell.

13. The measuring method as claimed in claim 12, wherein the gas interposed between the sensitive cell and the physiological liquid of the patient has a volume that is non-zero and less than or equal to 1 ml.

14. The measuring method as claimed in claim 12, wherein the measuring chamber is connected to a device for connection to the physiological liquid which includes cerebrospinal fluid of a patient.

15. The measuring method as claimed in claim 12, wherein the first physiological parameter corresponds to a pressure of the physiological liquid of the patient.

16. The measuring method as claimed in claim 12, comprising a determination step of determining at least a second physiological parameter through the measurement of the first physiological parameter.

17. The measuring device as claimed in claim 2, wherein the measuring chamber has an internal volume that is less than 0.7 ml.

18. The measuring method as claimed in claim 13, wherein the gas interposed between the sensitive cell and the physiological liquid of the patient has a volume that less than 0.7 ml.
